(19) United States
(12) Patent Application Publication
(10) Pub. No.: US 2023/0190686 A1
(43) Pub. Date: Jun. 22, 2023

(54) COMPOSITION AND METHOD FOR TREATMENT OF DIABETES

(71) Applicant: BIOKIER, INC., CHAPEL HILL, NC (US)

(72) Inventor: Jerzy Ryszard Szewczyk, Chapel Hill, NC (US)

(73) Assignee: BIOKIER, INC., CHAPEL HILL, NC (US)

(21) Appl. No.: 17/554,076

(22) Filed: Dec. 17, 2021

Related U.S. Application Data
(63) Continuation-in-part of application No. 17/527,401, filed on Jan. 1, 0001.

(51) Int. Cl. A61K 31/19; A61K 9/00; A61P 3/06

(52) U.S. Cl. CPC A61K 31/19; A61K 9/0056; A61P 3/06

(57) ABSTRACT

A method of treating a lipid related disease by oral delivery of butyric acid to the colon for release from about 5 and about 20 hours.

This application is a Continuation-In-Part of U.S. non-provisional application Ser. No. 17/527,401, filed on Nov. 16, 2021, and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a novel method and composition for treating lipid related diseases such as high cholesterol, high triglycerides, NASH, and related inflammation, in a manner that induces secretion of endogenous gut hormones.

### Description of Related Art

It has been disclosed that delivering butyric acid directly to the colon for immediate release helps with lipid related diseases.

Non-alcoholic steatohepatitis (NASH) is an advanced form of non-alcoholic fatty liver disease (NAFLD). NAFLD is caused by buildup of fat in the liver. When this buildup causes inflammation and damage, it is known as NASH, which can lead to scarring of the liver.

Cholesterol is a waxy substance found in blood. The body needs cholesterol to build healthy cells, but high levels of cholesterol can increase the risk of heart disease. With high cholesterol you can develop fatty deposits in your blood vessels. Eventually, these deposits grow, making it difficult for enough blood to flow through your arteries. Sometimes, those deposits can break suddenly and form a clot that causes a heart attack or stroke. High cholesterol can be inherited, but it's often the result of unhealthy lifestyle choices, which make it preventable and treatable. A healthy diet, regular exercise, and sometimes medication can help reduce high cholesterol.

Triglycerides are a type of fat (lipid) found in blood. The body converts any calories it doesn't need to use right away into triglycerides. The triglycerides are stored in your fat cells. Later, hormones release triglycerides for energy between meals. If you regularly eat more calories than you burn, particularly from high-carbohydrate foods, you may have high triglycerides (hypertriglyceridemia). High triglycerides may contribute to hardening of the arteries or thickening of the artery walls (arteriosclerosis)—which increases the risk of stroke, heart attack, and heart disease. Extremely high triglycerides can also cause acute inflammation of the pancreas (pancreatitis).

Butyric acid is a naturally occurring fatty acid occurring in the form of esters in animal fats and plant oils. For example, tributyrin, the triglyceride of butyric acid makes up three percent to four percent of butter. It is found in rancid foods, such as rancid butter and rancid cheese, and has a very unpleasant smell and taste. It is an important member of the fatty acid sub-group called the short-chain fatty acids.

The above naturally occurring product is difficult to administer, especially because taste of these products is extremely unpalatable and they are easily degraded in the digestive tract and/or absorbed.

There are obviously a number of compositions designed to deliver a medicament to the colon and to control the time of release. Such compositions include the three-component matrix structures such as disclosed in U.S. Pat. No. 7,431,943, to Villa et al., issued Oct. 7, 2008, and incorporated herein in its entirety by reference.

A number of other formulations are available for delivery of any desired compositions to the colon in a timed-release manner, including amylose-coated tablets, enterically-coated chitosan tablets, matrix-within-matrix or multimatrix systems, or polysaccharide-coated tablets. One example of a multimatrix controlled-release system is disclosed in U.S. Pat. No. 7,431,943, issued Oct. 7, 2008, to Villa et al., and incorporated herein by reference. Disclosed is a matrix-within-matrix design wherein a lipophilic phase and amphiphilic phase are incorporated within the inner matrix and at least a portion of the active ingredient is incorporated into the amphiphilic phase.

### BRIEF SUMMARY OF THE INVENTION

The present invention relates to the discovery that butyric acid compositions can be delivered orally by bypassing the stomach, duodenum, jejunum and ileum and releasing in the colon over a period selected from about 5 hours to about 20 hours is more effective at a lower dose than butyric acid compositions given directly to the stomach or colon for instant release. This approach can be used to treat lipid related diseases and is significantly more effective than giving butyric acid for immediate release in the colon.

In one embodiment of the present invention, there is an oral pharmaceutical composition for use in a human for the treatment of a lipid related disease comprising:
a) a single agent for inducing release of a gut hormone from an L-cell, wherein the single agent is butyric acid in an amount from about 70 mg to about 5 g at a rate of about 5% or less of the effective dose given directly to the stomach;
   wherein the butyric acid is formulated for delivery to and release in the colon over a period from about 5 to about 20 hours using a colon-targeted delivery system, which bypasses the stomach, duodenum, jejunum and ileum.

In another embodiment of the present invention, there is a method of treating a lipid related disease in a human comprising:
a) selecting a single agent causing gut hormone secretion from L-cells, wherein the agent is butyric acid in an amount from about 70 mg to about 5 g at a rate of about 5% or less of the effective dose given directly to the stomach;
b) wherein the butyric acid is formulated for delivery to and release in the colon over a period from about 5 to about 20 hours using a colon-targeted delivery system, which bypasses the stomach, duodenum, jejunum and ileum; and
c) orally administering the composition to the human.

### DETAILED DESCRIPTION OF THE INVENTION

This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “treating” refers to alleviating the specified condition, eliminating or reducing the symptoms of the condition, slowing or eliminating the progression of the condition, and preventing or delaying the initial occurrence of the condition in a subject, or preventing or delaying the reoccurrence of the condition in a previously afflicted subject.

As used herein a “condition” or “disorder” refers to Diabetes Type 2 and hyperlipidemia in a mammal, such as a human, or the like, to which an increase in the production of a gut hormone from L-cells during a meal would have a positive effect on the mammal.

The gut hormone secretion in the present invention is stimulated in L-cells present in the colon. This action can be partially or severely inhibited when treating a lipid related disease, and thus helps improve the treatment of the condition when using these types of compositions. While such L-cells are present in other parts of the digestive tract and other parts of the organism, they have the highest concentration in the colon. Stimulation of L-cells in the colon results in the most effective production of gut hormones possible, and thus the most effective treatment. Gut hormones from L-cells of the present invention include, but are not limited to, GLP-1, GP-2, PYY, and oxyntomodulin. Incretins such as GLP-1, in particular, are a gut hormone of interest in one embodiment. It has been discovered that instead of instant release in the colon, picking a release time from about 5 hours to about 20 hours is more effective than immediate release in the colon for lowering lipids.

The compounds of the invention for stimulating gut hormone release are natural compounds selected from the group comprising butyric acid. It is understood that this includes combinations of the compounds as well as each compound individually.

As used herein, “a compound” of the present invention includes all compounds described herein.

As used herein, “portal circulation” refers to the circulation of blood to the liver from among others, the right half of the colon, where gut hormone secretion takes place through the portal vein. Almost all of non-insulin related antidiabetic activity of GLP-1 are caused by activation of GLP-1 receptors in the portal system, resulting in improved glucose disposal and stimulation of the vagal nerves, and regulating central mechanism of metabolic control.

The compounds of the present invention may crystallize in more than one form, a characteristic known as “polymorphism”, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain compounds described herein contain one or more chiral centers, or may otherwise be capable of existing as multiple stereoisomers. The scope of the present invention includes mixtures of stereoisomers, as well as purified enantiomers or enantiomerically/diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof, in which one or more chiral centers are inverted.

Typically, but not absolutely, the compounds herein include the salts of the present compositions and include the pharmaceutically acceptable salts. Salts encompassed within the term “pharmaceutically acceptable salts” refer to non-toxic salts of the compounds of this invention. Salts of the compounds of the present invention may include acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, thethiodide, thmethylammonium, and valerate salts. Other salts, which are not pharmaceutically acceptable, may be useful in the preparation of compounds of this invention and these should be considered to form a further aspect of the invention.

As used herein, “delivery to the colon” refers to the oral administration of a composition of the present invention, wherein active compositions are delivered to the colon and released there, bypassing the stomach and the rest of the upper intestine. The composition is designed to release from about 5 to about 20 hours, though the time to entirely release can be about 10 hours. Numerous coatings are well known to deliver drugs just to the colon. As described elsewhere herein, the compounds are formulated so as to be taken orally and delivered to the colon by bypassing the stomach and upper intestine.

The “administering” of a composition of the present invention can refer to oral, and is not dependent on any particular means of administration other than delivery to the colon as intact compositions. One skilled in the art would be able to determine the exact amount (i.e., about less than 1 to about 5%) of the dose normally delivered to the stomach to some extent to the individual involved in therapy with the present invention, but always much less than the stomach dose. An effective amount of the L-cell stimulating composition is 70 mg to 5 g in a simultaneously administered composition. The exact amount being about 5% or less of the hypothetical stomach dose 100 g or more.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means the amount which, as compared to a corresponding subject who has not received such an amount, results in improved treatment, healing, prevention, amelioration of a disease, disorder, side effect, or a decrease in the rate of advancement of a disease or disorder at a dose of about 5% or less of the dose designed to be delivered to the stomach. The term also includes within its scope the amounts effective to enhance normal physiological function. A therapeutically effective amount will produce a “therapeutic effect”.

For use in therapy, therapeutically effective amounts of a compound of the present invention, as well as salts thereof, are presented as a pharmaceutical composition formulated to release in a colon-targeted delivery system.

The present invention provides pharmaceutical compositions that include effective amounts of a compound as herein described, or a salt thereof, and one or more pharmaceutically acceptable carriers, diluents, or excipients. The carrier(s), diluent(s), or excipient(s) must be acceptable, in the sense of being compatible with the other ingredients of the formulation and not deleterious to the recipient of the pharmaceutical composition, and consistent with the mode of administration (i.e., oral or rectal).

In accordance with another aspect of the invention, there is also provided a process for the preparation of a pharmaceutical formulation, including admixing a compound of the present invention or salts thereof, with one or more pharmaceutically acceptable carriers, diluents, or excipients.

A therapeutically effective amount of a compound of the present invention will depend upon a number of factors. For example, the species, age, and weight of the recipient, the precise condition requiring treatment and its severity, the nature of the formulation, and the type of colon-targeted delivery system are all factors to be considered. The therapeutically effective amount ultimately should be at the discretion of the attendant, physician, or veterinarian. Regardless, an effective amount of butyric acid of the present invention for the treatment of humans suffering from a lipid related disease such as high cholesterol, both LDL and total, postprandial triglycerides, lower liver enzymes such as Alanine Aminotransferase (ALT) and aspartate aminotransferase (AST), and hypercholesterolemia, an overweight condition and associated conditions, generally, should be in the range of 0.1 mg/kg to 70 mg/kg body weight of recipient (mammal) per day. More frequently, the effective amount should be in the range of 1 mg/kg to 70 mg/kg per body weight per day. Thus, for a 70 kg adult mammal, the actual amount per day would usually be from 70 mg to 5 g. This amount may be given in a single dose per day or in a number (such as two, three, four, five, or more) of sub-doses per day such that the total daily dose is the same. An effective amount of a salt or solvate thereof may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.25 mg to 1 g of a butyric acid composition. Incretin-stimulating compounds of the present invention's dosage depends on the lipid related disease being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

The compounds of the present invention, or a salt thereof, are administered by a targeted drug delivery system. In one embodiment, the delivery systems may be employed for targeting drug delivery to the colon and bypassing the stomach and small intestine. Such drug delivery systems include covalent linkage compositions, polymer-coated compositions, compositions embedded in matrices, time released compositions, redox-sensitive polymer compositions, bioadhesive compositions, micropartical coating compositions, and osmotic delivery compositions. Suitable compositions include those containing polysaccharides, such as chitosan, pectin, chondroitin sulphate, cyclodextrin, dextrans, guar gum, inulin, amylase, and locust bean gum. The compounds may also be coupled with soluble polymers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palm itoyl residues. Furthermore, the compounds may be coupled to a class of biodegradable polymers; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. Those of particular effectiveness in the present invention include embodiments of multimatrix targeted systems. Of particular effectiveness in the present invention are the targeted matrix-in-matrix systems comprising a formulation of a hydrophilic first matrix, comprising a lipophilic phase and an amphiphilic phase, wherein the lipophilic phase and the amphiphilic phase are in a second matrix together and the second matrix is dispersed throughout the hydrophilic first matrix, and wherein the pharmaceutical composition containing the compound is at least partially incorporated into the amphiphilic phase. Examples of some of the matrix-in-matrix formulations are disclosed in U.S. Pat. No. 7,431,943, as noted above. Those skilled in the art will appreciate the use of such compositions for the purposes of targeting delivery of the compounds of the present invention, or a salt thereof, to the colon of the subject being treated. The methods for the formulation of such compositions for targeted delivery are within the skill of the art, in view of this disclosure.

The compounds of the present invention, or a salt thereof, may be employed alone or in combination with other therapeutic agents. In one embodiment, they are combined with other agents useful for the treatment of lipid related diseases. The compound(s) of the present invention and the other pharmaceutically active agent(s), may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration, in combination of a compound of the present invention or a salt, or solvate thereof, with other treatment agents may be in combination by administration concomitantly in: (1) a unitary pharmaceutical composition including both compounds; or (2) separate pharmaceutical compositions, each including one of the compounds. Alternatively, the combination may be administered separately in a sequential manner wherein one treatment agent is administered first and the other second, or vice versa. Such sequential administration may be close in time or remote in time.

The compounds of the present invention may be used in the treatment of a lipid related disease. As such, the compounds of the present invention may be used in combination with a variety of other therapeutic agents useful in the treatment of this condition. As discussed briefly above, current therapies include diet, exercise, statins, niacin, fat absorption inhibitors. The compounds of the present invention may be combined with these or other medical therapies to treat a lipid related disease and/or prevent associated disorders and conditions.

### EXAMPLES

### Example 1

Sustained-release (5-20 hours), colon-targeted tablets containing 500 mg of butyric acid sodium salt were made as described in (BioKier patent for additional coat under Phloral). Patients with high cholesterol LDL over 100 mg/dL and/or total cholesterol over 200 mg/dL, including those already treated with statins, were dosed for 28 days with 1-3 tablets BID. After 28 days of dosing with colon-targeted tablets total and LDL cholesterol were measured and fond to be significantly lower (by 20-over 40%) than before treatment. In addition, triglyceride, liver enzymes ALT (alanine transaminase) and AST (aspartate aminotransferase) were also lowered. Some patients also reported reduced appetite.

The following references are included in the application by reference in their entirety.
1. Mayo Clin. Proc., 1993, Vol 68, 978, incorporated herein by reference,
2. U.S. Pat. No. 7,431,943 B1, incorporated herein by reference,
3. Diabetes, Obesity and Metabolism, 9 (Suppl. 1), 2007, 23-31, incorporated herein by reference,
4. Toft-Nielsen M B, Damholt M B, Madsbad S et al., Determinants of the impaired secretion of glucagon-like peptide-1 in type 2 diabetic patients., J Clin Endocrinol Metab, 2001; 86:3717-3723,
5. Rask E, Olsson T, Soderberg S et al., Impaired incretin response after a mixed meal is associated with insulin resistance in nondiabetic men., Diabetes Care, 2001; 24:1640-1645,
6. Provisional patent applications (BIOK001PR) 61/143,951, filed Jan. 12, 2009, and (BIOK001PR-C) 61/293,773, filed Jan. 11, 2010, incorporated herein in their entirety by reference; and
7. BIOK001-C-PCT application number PCT/US2010/020629, incorporated herein in its entirety by reference.

What is claimed is:

1. An oral pharmaceutical composition for use in a human for the treatment of a lipid related disease comprising:
a) a single agent for inducing release of a gut hormone from an L-cell, wherein the single agent is butyric acid in an amount from about 70 mg to about 5 g at a rate of about 5% or less of the effective dose given directly to the stomach;
wherein the butyric acid is formulated for delivery to and release in the colon over a period from about 5 to about 20 hours using a colon-targeted delivery system, which bypasses the stomach, duodenum, jejunum and ileum.

2. A method of treating a lipid related disease in a human comprising:
a) selecting a single agent causing gut hormone secretion from L-cells, wherein the agent is butyric acid in an amount from about 70 mg to about 5 g at a rate of about 5% or less of the effective dose given directly to the stomach;
b) wherein the butyric acid is formulated for delivery to and release in the colon over a period from about 5 to about 20 hours using a colon-targeted delivery system, which bypasses the stomach, duodenum, jejunum and ileum; and
c) orally administering the composition to the human.
